(19) United States
(12) Patent Application Publication
(10) Pub. No.: US 2013/0230910 A1
(43) Pub. Date: Sep. 5, 2013

(54) Apparatus and Method for Process Challenge Devices

(71) Applicants: Dennis E. Christensen, Sunnyvale, CA (US); R. Daniel Webster, Sunnyvale, CA (US)

(72) Inventors: Dennis E. Christensen, Sunnyvale, CA (US); R. Daniel Webster, Sunnyvale, CA (US)

(73) Assignee: Process Challenge Devices, LLC., Sunnyvale, CA (US)

(21) Appl. No.: 13/783,844

(22) Filed: Mar. 4, 2013

Related U.S. Application Data
(60) Provisional application No. 61/606,241, filed on Mar. 2, 2012.

(51) Int. Cl. C12Q 1/22

(52) U.S. Cl. CPC C12Q 1/22
USPC 435/287.4; 29/592; 29/426.2

(57) ABSTRACT

A process challenge device tailored to mimic the resistance of a particular product to a particular biological inactivation, disinfection, or sterilization process, and used to challenge the process, thus providing a means to validate the efficacy of the process. The process challenge device is used by subjecting the device containing indicator organisms to an inactivation or sterilization process, and culturing any surviving indicator organisms as a means to assess the efficacy of procedures for the inactivation of microorganisms. The device uses a self-containing biological indicator (SCBI) with a biological indicator and media ampule located within a plastic vial. By altering the materials and/or configuration of the SCBI a wide range of resistances to sterilant gas processes may be achieved.

### CROSS REFERENCE TO OTHER APPLICATIONS

This application claims priority of U.S. Provisional Patent Application No. 61/606,241, filed Mar. 2, 2012, which is hereby incorporated by reference in its entirety.

### FIELD OF THE INVENTION

This invention relates generally to process challenge devices, in particular to process challenge devices using process indicators such as biological indicator organisms or biological enzymes sealed in containers made from specially chosen materials, used to assess the efficacy of procedures for the inactivation of microorganisms in industries related to health care, food packaging and preparation, and other industries that use biological indicators for sterility assurance testing. Specifically this invention relates to modification of existing Self Contained Biological Indicators (SCBIs) to achieve variable resistance to gaseous/vapor sterilization.

### BACKGROUND OF THE INVENTION

There are several conventional methods to test the effectiveness of a given gas or vapor sterilization, disinfection, or biological inactivation process (hereafter referred to collectively as “inactivation process”). A first method is to inoculate a sample product with a known quantity of a specific indicator organism (the “inoculate”), subject the inoculated product to the appropriate process, recover the sample inoculate, and culture the inoculate in a specific growth medium to determine whether there were any surviving organisms. A second method is to use a biological indicator which is inoculated with a known quantity of a specific indicator organism, subject the biological indicator to the appropriate process, and culture the biological indicator to determine whether there are any surviving organisms. Typically, in both the first and second methods the absence of growth of the indicator organisms in the growth medium indicates a successful inactivation process. Direct inoculation of sample product is generally done during early validation of a biological inactivation process. Biological indicators are generally used to test repeat processing. A third method is to use an indicator enzyme, subject the enzyme to the appropriate process, and then test for enzyme activity. If no activity is indicated, it is presumed that living organisms would similarly be inactivated.

Currently there are three primary inactivation processes employed in the medical device and health care industries: steam, ethylene oxide gas, and ionizing radiation. Several other processes such as dry heat, hydrogen peroxide, chlorine dioxide, peracetic acid, ozone, and plasma are also in various stages of use and acceptance. Each of these inactivation processes except for ionizing radiation requires unique biological indicator organisms, growth media, and procedures to confirm sterilization effectiveness.

Process challenge devices containing Self Contained Biological Indicators (SCBIs) which do not require inoculation of a product are in regular use in contract sterilization and health care facilities (hospitals) The resistance of a process challenge device to a particular biological inactivation process is given as a D Value. This is defined as the exposure time in minutes required under a defined set of conditions to cause a 1-logarithm or 90% reduction in the population of a particular organism. Many process challenge devices have a single unchanging D Value. In order to create a variable resistance to the inactivation process experienced by actual product being processed, due to packaging of the product, the location of a product within a load being processed, or other factors, these devices must generally be wrapped or contained within packaging or other protective material similar to that used on the products being sterilized, so that the process challenge device is exposed to the same environment as the products being processed. Alternatively, in some cases the process challenge device is buried in the most protected location within a load being sterilized. Therefore, these devices cannot be used alone to validate a biological inactivation process without additional protection from the process to simulate the higher resistance of the actual products to the process.

For example, Welsh et al., U.S. Pat. No. 4,839,291, discloses a process challenge device that is composed of a number of elements including an outer tube and an inner tube assembled in a manner intended to create a tortuous path to impede the flow of sterilant to the biological indicator contained within the tubes, thereby creating a D Value. Typical of many prior art devices, the device of Welsh et al. is larger and more expensive to manufacture than the present invention, and its resistance to a particular sterilization process may not be easily and accurately varied. Additionally, the materials used may not be suitable for the newer inactivation processes, such as hydrogen peroxide, ozone, and plasma, because the sterilants used may destructively react with elements of the Welsh et al. process challenge device.

More recently, U.S. Pat. Nos. 5,942,408 and 6,651,096 to Christensen, et al. describe process challenge devices tailored to mimic the resistance of a particular product to a particular biological inactivation, disinfection, or sterilization process. The process challenge device is then used to challenge the process, thus providing a means to validate the efficacy of the process. The process challenge device is used by subjecting the device containing indicator organisms to an inactivation or sterilization process, and culturing any surviving indicator organisms as a means to assess the efficacy of procedures for the inactivation of microorganisms. The device includes a biological indicator organism stored on a carrier substrate enclosed within a chamber formed by a barrier film material. The specific indicator organism and carrier substrate are chosen for their appropriateness for a given process. The materials comprising the barrier film material of the process challenge device are chosen for the materials' specific resistance to the given process. U.S. Pat. Nos. 5,942,408 and 6,653,096 are hereby incorporated by reference in their entirety.

### SUMMARY OF THE INVENTION

The present invention provides a convenient, low cost device to challenge biological inactivation process effectiveness that may be easily constructed with variable resistance to a particular biological inactivation process by substitution of known materials having known resistances to particular inactivation process being used for sterilization. The present invention also provides for changes to the design configuration of SCBI components to achieve variable resistance.

The present invention is a process challenge device that has a biological indicator and growth media contained with a plastic vial or other suitable chamber. The cap of the vial has a barrier film which is selected to mimic the resistance of packaging of items that are being sterilized in various sterilization processes. The present invention is a process challenge device based on changing the configuration and/or component materials of an industry standard self-contained biological indicator (SCBI). These changes are directed at changing the resistance to ingress and removal of sterilant gas to the BI contained within a SCBI device. These methods include but are not limited to:
a. Substituting a membrane barrier film for the cap filter
b. Replacing the cap with a heat sealed membrane material
c. Changing the cap material and/or thickness
d. Changing the cap vent hole diameter and/or length
e. Replacing the cap with a plug having a variable hole diameter and/or length
f. Changing the vial material and/or thickness.

Domestic and international regulatory guidelines permit the use of biological process challenge devices that demonstrably have resistance to sterilization equal to or greater than the material or product and package combination to be subjected to the process, which process is being tested by the process challenge device. The “product package combination” refers to the characteristics of the product itself and of any associated packaging which may exist as these characteristics relate to or affect the product and package combinations resistance to a particular inactivation process. A product and package combination including no packaging is included in this definition.

By knowing the specific characteristics of the biological inactivation process to be tested, the film material or materials to be used in construction of the SCBI process challenge device may be chosen based on the known gas permeation, temperature and chemical resistance values of available film materials. This results in a SCBI which can be used alone as a process challenge device to mimic the resistance to the inactivation process experienced by the product being processed, rather than requiring that the process challenge device be processed within a sterilizer load, or with additional packaging or protections to simulate product resistance to the process.

In some embodiments, the process challenge device of the present invention may include process exposure indicators. Any means for visually indicating that the device has been exposed to the inactivation process may be used, however, a paper or polymer plastic label which is chemically treated to change color when the device has been exposed to the biological inactivation process is preferable.

### BRIEF DESCRIPTION OF THE DRAWINGS

FIG. 1A shows an example of the self-contained biological indicator (SCBI) device.

FIG. 1B is an exploded view of the SCBI of FIG. 1.

FIG. 2A is a top view of the cap of the SCBI.

FIG. 2B is a bottom view of the cap of the SCBI.

FIG. 3 shows the cap of the SCBI with the barrier film not yet inserted.

FIG. 4A shows a process challenge device using a substitute membrane barrier film for the cap filter.

FIG. 4B shows a process challenge device using a heat sealed membrane material attached to the top of the vial.

FIG. 4C shows a process challenge device using a heat sealed membrane material attached to the internal perimeter of the vial.

FIG. 4D shows a process challenge device where the vent hole in the cap has been altered.

FIG. 4E shows a process challenge device where the cap of the SCBI has been replaced with a plug having a variable hole diameter or length.

FIG. 4F shows a vial where the vial materials and thickness have been changed.

### DETAILED DESCRIPTION OF THE INVENTION

Biological inactivation process validation and verification of process effectiveness are important aspects of any inactivation process for medical devices or pharmaceuticals or any treatment process for sterilization, biological inactivation or disinfection of food products.

Conventional means to test the effectiveness of a given inactivation process require the inoculation of a sample product with a known quantity of a specific indicator organism (the “inoculate”), subjecting the inoculated product to the appropriate process, recovering the sample inoculate, and culturing the inoculate in an appropriate growth medium to determine whether any indicator organisms survive.

The international guidelines for sterilization of health care products allows for the use of a process challenge device as an alternative to the conventional method of process validation, and biological inactivation verification, described above. In methods using a process challenge device, the process challenge device is cycled through the process with the products and then separately analyzed to determine the efficacy of the process. The process challenge device must be as resistant or more resistant, to the inactivation process than the product-package combination being sterilized. Normally this requires that the process challenge device be protected within actual product being processed, or be otherwise protected in order to mimic the resistance of the product to the process.

The present invention is a process challenge device in which the resistance of the SCBI is controlled by changing the materials, component configurations and/or dimensions of the SCBI.

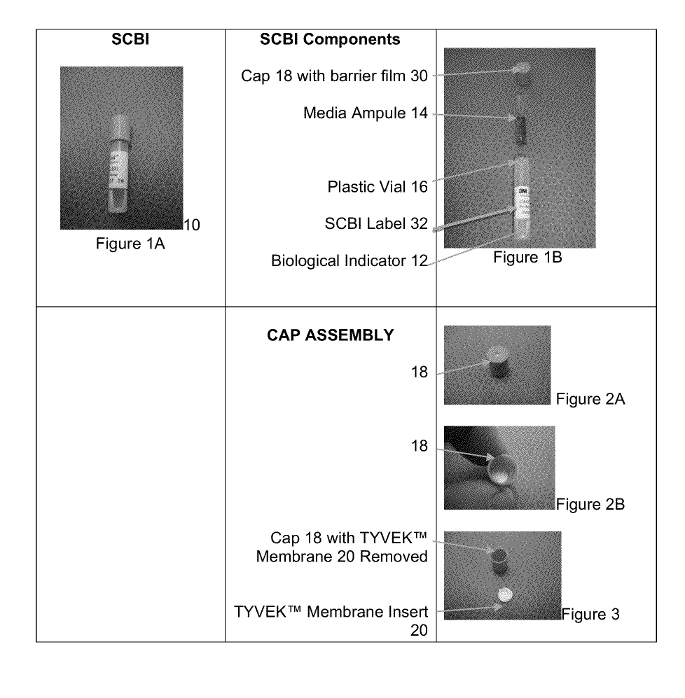

FIGS. 1A, 1B, 2A, 2B and 3 show an example of a typical self-contained biological indicator (SCBI) device 10. This example device 10 has a biological indicator 12 and a media ampule 14 contained within a plastic vial 16. The vial 16 has a cap 18 that covers and seals the vial 16. The cap 18 has a TYVEK™ cap filter membrane 20 affixed therein.

The process challenge device is created by modification of the SCBI 10 such that the characteristics of the vial 16, chamber with the vial 16, cap 18 or sealing mechanism are altered to achieve a desirable sterilization resistance. A few of the possible modifications envisioned are discussed below.

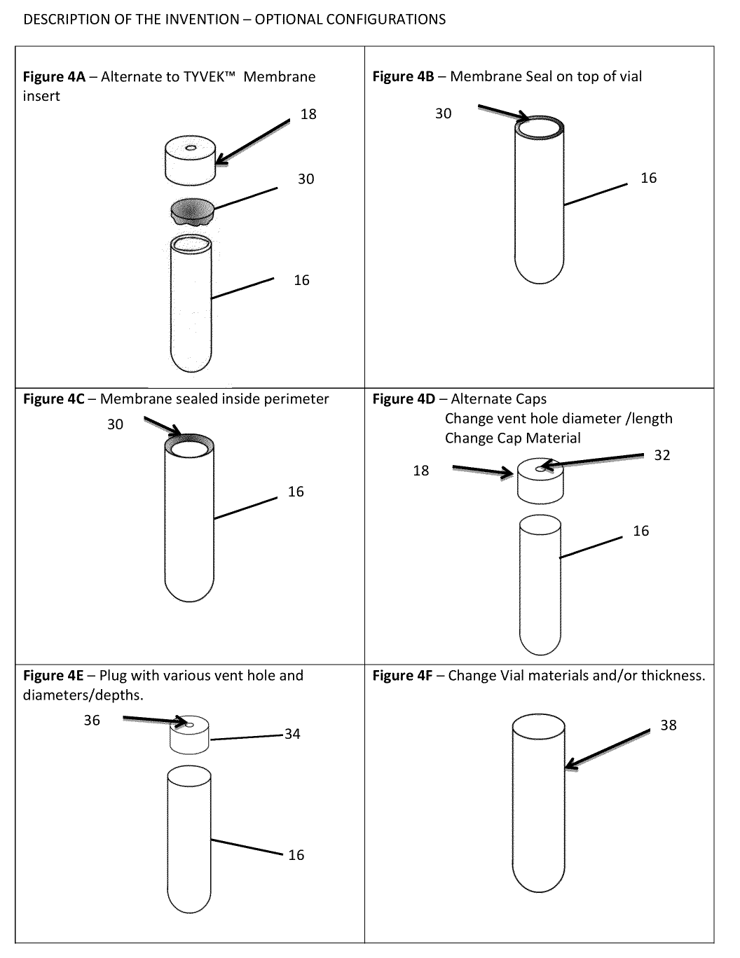

FIG. 4A shows a process challenge device using a substitute membrane barrier film 30 for the cap 18 filter. In this embodiment, the TYVEK™ film 20 can be removed a new membrane 30 having more resistance to sterilizing gases is placed in the cap 18.

FIGS. 4B and 4C show process challenge devices using a membrane material 30 sealed to the vial 16. One option for sealing the membrane to the vial is by heat sealing. Alternately, adhesive or other attachment mechanism may be used. This may be done in any convenient configuration. FIG. 4B shows the membrane 30 attached to the top of the vial 16. FIG. 4C shows the membrane 30 attached to the internal perimeter of the vial 16.

FIG. 4D shows a process challenge device where the vent hole 32 in the cap 18 has been altered. The alteration may include increasing the size of the hole 32, adding material to the hole 32 to decrease the size of the hole, adding thickness to the cap 18 to lengthen the hole 32 or removing thickness of the cap 18 to decrease the length of the hole 32. The cap 18 may be press-sealed or otherwise sealed to the vial 16.

FIG. 4E shows a process challenge device where the cap 18 of the SCBI 10 has been replaced with a plug 34. The plug 34 may include a hole 36 having a length and diameter selected for their action in providing a barrier to the sterilization process. In some cases, the plug 34 may have a hole 36 that is variable in diameter, length or both. The plug 34 may be press-sealed or otherwise sealed to the vial 16.

FIG. 4F shows a vial 38 where the vial 38 materials and thickness have been changed. Other variations may use a change to the material or the thickness alone as desired.

Alternate versions may also use a similar appearing cap, but the new cap having altered properties to achieve an appropriate resistance, this may be done by using specific materials and configuration of openings. Other modifications may be made in the same spirit to create a device that alters the resistance of a product to sterilization. Alternately, the device may be constructed during initial manufacturing in a similar configuration to any of the above embodiments.

The process challenge device can be constructed with a resistance to a particular biological inactivation process at least as great as the resistance of the product typically processed, so that the process challenge device may be used alone to test the inactivation process, without accompanying materials or products. The adjustment of the process challenge device may be accomplished by selection of the biological indicator and barrier film.

The biological indicator may be of any suitable form. Examples of biological indicator include but are not limited to a cellulose disk inoculated with a known quantity of a biological indicator organism which may be one of several types chosen for the organism's appropriateness to the method of biological inactivation to be used. For ethylene oxide gas sterilization, the recommended biological indicator has a 10.sup.6 (microorganisms per indicator) concentration of Bacillus atropheus, typically on a cellulose substrate. The international guidelines for steam sterilization recommend a biological indicator with a 10.sup.6 concentration of Bacillus stearothermophilus. For food processing a biological indicator inoculated with Clostridium is preferred. Other preferred microorganisms include Bacillus circulans, Bacillus cereus, and Bacillus Pumilus. In still other embodiments, other process indicators, such as a biological enzyme, could be used.

Other newer types of inactivation processes, such as hydrogen peroxide or ozone plasma may be reactive with the cellulose carrier typically used to carry biological organisms. Therefore, for these processes, a carrier other than cellulose should be used for process indicator, such as a fibrous polyester substrate, a porous ceramic, fiber glass, or a substrate composed of plastics such as microporous polymeric compounds including polypropylene, polyethylene, and polysulfone, or a nonporous inorganic substrate such as a metal, glass or fiberglass. For example, in a process challenge device embodiment for hydrogen peroxide sterilization, the process indicator preferably has a 10.sup.6 concentration of Bacillus stearothermophilus on a non-reactive carrier comprising a microporous filter medium, preferably of a non-reactive polymer such as polypropylene, polyethylene or polysulfone. In alternate embodiments, other desirable microorganisms may be used.

The membrane film material of the SCBI process challenge device is designed to create a specific resistance greater than or equal to that of the material or product package combination being treated by a specific process, and any related packaging associated with the material. The process resistance of the challenge device is determined by the properties of the barrier film material or materials chosen. Such properties include gas permeability, temperature and chemical resistance.

Suitable candidate materials for the membrane film material include, but are not limited to, polymer film materials, such as polyolefins (e.g. polyethylene or polypropylene), polyesters (e.g. polyethylene terephthalate (MYLAR)), polybutylene terephthalate, or PETG copolyester, polyamides (nylons), vinyl-chloride polymers, polyvinylidene chloride (e.g. SARAN), polyvinylidene flouride, polyamides, ethylene-vinyl acetate, ethylene vinyl alcohol, aluminized polyester, etc., or nonpolymer films, such as aluminum foil, silica oxide and alumina oxide, either separately or in combination. Multilayer films which are laminated with adhesive or formed by coextrusion may also be used. The specific materials and conformation chosen will vary depending on the characteristics of the inactivation process in which the process challenge device will be used.

The media ampule 14 contains a test or culture medium. Depending on the culture medium and the inactivation process chosen, the ampule may be formed of a more resistant barrier material in order to protect culture medium from the biological inactivation process. If for example, the chosen culture medium is reactive to the sterilizing agent used in gas sterilization, a gas impermeable barrier material such as glass is typically used for the body of the ampule 14.

The culture medium may be of any suitable type. For example a liquid culture medium such as a soybean casein digest medium or the like is suitable. In alternative embodiments, a gelatin medium could be used. In embodiments using a process indicator comprising a biological enzyme, the test medium may be an appropriate enzyme substrate. Other suitable media may be used depending on the biological indicator used.

In some embodiments, the process challenge device of the present invention may include process exposure indicators. Any means for visually indicating that the device has been exposed to the inactivation process may be used. For example, a paper label 32 that is chemically treated to change color when the device has been exposed to the biological inactivation process may be used. A variety of such exposure indicators are commercially available.

In use, for either process validation or verification, a single SCBI process challenge device constructed as discussed above is used. Alternatively, one or more process challenge devices are placed at various locations within a sterilizer load or processing batch, preferably on the exterior of the product packaging at different locations within the load.

Time is saved by not having to inoculate sample products before they are packaged and no actual packaged products have to be sacrificed. The load is then subjected to the chosen sterilization or inactivation cycle or other applicable process. After the process cycle, the process challenge devices are removed and may be taken to a laboratory where the biological indicator is incubated in a suitable media.

Alternately, in embodiments with the media ampule, a local incubator or other controlled temperature chamber may be used since everything needed for culturing the indicator organism is contained within the process challenge device. In this embodiment, a crushable ampule contains a liquid media. Once the processing of the batch is complete, the ampule is crushed allowing self-contained incubation of the biological indicator. The media may be selected to change color if growth is indicated. Other indication mechanisms may also be used.

Significant savings are realized by using this embodiment of the SCBI process challenge device because minimal laboratory facilities and personnel are required to analyze the results of each process challenge device. For example, time is saved in not having to prepare culture medium or culture tubes in which to incubate the indicator organisms, thereby avoiding the need for a complete laboratory. By eliminating many of the laboratory procedures, the level of training needed for most of the process steps of inactivation process validation or process verification are also reduced. Exposure of personnel to the indicator organisms and to any residual sterilizing agent is also reduced. The absence of growth of indicator organisms indicates a successful sterilization, biological inactivation process, or disinfection process.

The preferred embodiments described herein are illustrative only and although the examples given include many specificities, they are intended as illustrative of several possible embodiments of the invention. Other embodiments and modifications will, no doubt, occur to those skilled in the art. Thus, the examples given should only be interpreted as illustrations of some of the preferred embodiments of the invention, and the full scope of the invention should be determined by the appended claims and their legal equivalents.

What is claimed is:

1. A process challenge device for testing the efficacy of sterilization, the process challenge device comprising:
a self-contained biological indicator including:
a media ampule,
a biological indicator,
a vial containing said media ampule and biological indicator,
and a sterilization cap covering an opening in the vial,
wherein characteristics of said cap are selected to create a resistance to a selected sterilization process.

2. The process challenge device of claim 1, further comprising an original cap, wherein said original cap is removed and replaced with said sterilization cap.

3. The process challenge device of claim 1, wherein the sterilization cap has an opening therethrough, said opening sized and shaped to provide a selected resistance to a sterilization process.

4. The process challenge device of claim 1, wherein the sterilization cap is a membrane barrier film sealed to the vial.

5. A method of creating a process challenge device, the method comprising:
(a) obtaining a self-contained biological indicator having a biological indicator and a media ampule located within a chamber;
(b) and modifying the chamber to achieve a selected sterilization process resistance.

6. The method of claim 5, wherein step (b) is achieved by substituting a membrane barrier film for an original cap filter located in a cap of the chamber.

7. The method of claim 6, wherein the membrane barrier film has greater resistance to sterilizing gases than the original cap filter.

8. The method of claim 6, wherein the membrane barrier film is sealed to a top of the chamber.

9. The method of claim 6, wherein the membrane barrier film is sealed to an internal perimeter of the chamber.

10. The method of claim 5, wherein step (b) is achieved by altering a vent hole in a cap of the chamber.

11. The method of claim 10, wherein a thickness of the cap is altered.

12. The method of claim 10, wherein a size of a hole in the cap is altered.

13. The method of claim 10, wherein the cap is press-sealed to an opening in the chamber.

14. The method of claim 5, wherein step (b) is achieved by removing a cap of the chamber and replacing the cap with a plug.

15. The method of claim 14, wherein the plug has a hole with a length and diameter selected to provide a specific resistance to the sterilization process.

16. The method of claim 14, wherein the hole has a variable length.

17. The method of claim 14, wherein the hole has a variable diameter.

18. The method of claim 5, wherein the chamber is formed of an original material and wherein step (b) is achieved by replacing the original material with a second material.

19. The method of claim 5, wherein the chamber is formed of a material with a thickness and wherein step (b) is achieved by altering the thickness of the material.
